(19) United States
(12) Patent Application Publication
(10) Pub. No.: US 2024/0315714 A1
(43) Pub. Date: Sep. 26, 2024

(54) INTRAVASCULAR CROSSING AND ATHERECTOMY APPARATUSES AND METHODS OF USE

(71) Applicant: Bard Peripheral Vascular, Inc., Franklin Lakes, NJ (US)

(72) Inventors: Kyle A. Thomas, Phoenix, AZ (US); Genevieve Messina, Mesa, AZ (US); Amanda Young, Phoenix, AZ (US); Keith S. Harris, Mesa, AZ (US); Clint Solomon, Gilbert, AZ (US)

(73) Assignee: Bard Peripheral Vascular, Inc., Franklin Lakes, NJ (US)

(21) Appl. No.: 18/573,528

(22) PCT Filed: Jun. 23, 2021
(86) PCT No.: PCT/US2021/038680

§ 371 Date: Dec. 22, 2023

(51) Int. Cl. A61B 17/22; A61B 17/00; A61B 34/00

(52) U.S. Cl. CPC A61B 17/22012; A61B 34/73; A61B 2017/00292; A61B 2017/00778; A61B 2017/22025; A61B 2017/22038; A61B 2017/22069; A61B 2017/22079; A61B 2017/22094; A61B 2034/731

(57) ABSTRACT

An intravascular apparatus includes an electrical circuit configured to generate a series of electrical pulses. An intravascular catheter includes an elongate catheter body having a distal end portion and a distal end at a distal terminus of the distal end portion. A solenoid-type reciprocating device is located at the distal end portion of the elongate catheter body. The solenoid-type reciprocating device has a coil and a distal tip member. The coil is electrically coupled to the electrical circuit. The distal tip member has a proximal armature section and a distal working end section. The coil defines a coil aperture that is configured to slidably receive the proximal armature section. The proximal armature section is configured to axially reciprocate relative to the coil when the coil is energized.

### TECHNICAL FIELD

The present specification generally relates to intravascular crossing and atherectomy apparatuses and methods of use and, more specifically, to intravascular crossing and atherectomy apparatuses that utilize repeated impacts to break up and aspirate plaque.

### BACKGROUND

Atherosclerosis occurs when fat, cholesterol or other substances accumulate in walls of blood vessels. Atherectomy is a minimally invasive endovascular surgery technique for removing atherosclerotic build-up from blood vessels within the body. An atherectomy-type catheter may be used for the purpose of breaking-up and removing plaque from calcified vessels that have stenosis.

Chronic total occlusions (CTO) occur when a blood vessel is completely blocked. Current CTO devices create a narrow channel through the lesion that allow for subsequent atherectomy or balloon dilation to expand the vessel lumen. Calcified end caps are often challenging for CTO devices to cross and treat. Many current devices work well in calcified plaque or thrombus/fibrous plaque, but may have difficulties in other plaque morphologies.

Accordingly, here is a need for intravascular crossing and atherectomy apparatuses and methods of use that can be used to target hard areas of plaque in order to cross challenging calcified vessels.

### SUMMARY

According to a first embodiment, an intravascular apparatus includes an electrical circuit configured to generate a series of electrical pulses. An intravascular catheter includes an elongate catheter body having a distal end portion and a distal end at a distal terminus of the distal end portion. A solenoid-type reciprocating device is located at the distal end portion of the elongate catheter body. The solenoid-type reciprocating device has a coil and a distal tip member. The coil is electrically coupled to the electrical circuit. The distal tip member has a proximal armature section and a distal working end section. The coil defines a coil aperture that is configured to slidably receive the proximal armature section. The proximal armature section is configured to axially reciprocate relative to the coil when the coil is energized by the series of electrical pulses generated by the electrical circuit, and the distal working end section is configured to axially reciprocate from the distal end of the elongate catheter body, in unison with the axial reciprocation of the proximal armature section.

In another embodiment, an intravascular catheter includes an elongate catheter body that has a distal end portion and a distal end at a distal terminus of the distal end portion. A solenoid-type reciprocating device is located at the distal end portion of the elongate catheter body. The solenoid-type reciprocating device has a coil and a distal tip member. The distal tip member has a proximal armature section and a distal working end section. The coil defines a coil aperture configured to slidably receive the proximal armature section. The proximal armature section is configured to axially reciprocate relative to the coil and the distal working end section is configured to axially reciprocate from the distal end of the elongate catheter body.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 diagrammatically depicts an intravascular apparatus that includes an intravascular catheter including a solenoid-type reciprocating device, according to one or more embodiments shown and described herein;

FIG. 2 is a diagrammatic section view of a distal end portion of the intravascular catheter of FIG. 1 including the solenoid-type reciprocating device, according to one or more embodiments shown and described herein;

FIG. 3 is a diagrammatic perspective view of a distal working end section of a distal tip member for the solenoid-type reciprocating device of FIG. 2, according to one or more embodiments shown and described herein;

FIG. 4 is an end view of the distal working end section of FIG. 3, according to one or more embodiments shown and described herein;

FIG. 5 is a diagrammatic perspective view of another distal working end section of a distal tip member for the solenoid-type reciprocating device of FIG. 2, according to one or more embodiments shown and described herein;

FIG. 6 is a diagrammatic section view of the distal end portion of the intravascular catheter of FIG. 2, according to one or more embodiments shown and described herein;

FIG. 7 illustrates operation of the solenoid-type reciprocating device of FIG. 2 in a retracted configuration, according to one or more embodiments shown and described herein;

FIG. 8 illustrates operation of the solenoid-type reciprocating device of FIG. 2 in an extended configuration, according to one or more embodiments shown and described herein;

FIG. 9 illustrates operation of another solenoid-type reciprocating device in a retracted configuration, according to one or more embodiments shown and described herein;

FIG. 10 illustrates operation of the solenoid-type reciprocating device of FIG. 9 in an extended configuration, according to one or more embodiments shown and described herein; and

FIG. 11 is a diagrammatic section view of a distal end portion of an intravascular catheter including the solenoid-type reciprocating device, according to one or more embodiments shown and described herein.

### DETAILED DESCRIPTION

Embodiments described herein are generally directed to intravascular crossing and atherectomy apparatuses that include a power source with an electrical circuit and an intravascular catheter with an elongate catheter body and a solenoid-type reciprocating device that is used to break up plaque in calcified blood vessels. The solenoid-type reciprocating device is located at a distal end portion of the catheter body and has a coil electrically coupled to the electrical circuit and a distal tip member in the coil. The distal tip member has a proximal armature section and a distal working end section. The coil defines a coil aperture configured to slidably receive the proximal armature section such that the proximal armature section axially reciprocates relative to the coil when the coil is energized by a series of electrical pulses generated by the electrical circuit. The distal working end section is configured to axially reciprocate from a distal end of the catheter body in unison with the axial reciprocation of the proximal armature section.

Various aspects will now be described with reference to specific forms or embodiments selected for purposes of illustration. It will be appreciated that the spirit and scope of the atherectomy apparatuses disclosed herein are not limited to the selected forms. Moreover, it is to be noted that the figures provided herein are not drawn to any particular proportion or scale, and that many variations can be made to the illustrated embodiments. Brief introductions to some of the features, which are common to the described embodiments of the atherectomy apparatuses, are now described.

To assist in the description of these components of the atherectomy apparatuses, the following coordinate terms are used. A “longitudinal axis” is generally parallel to the elongated axis of the coil aperture disclosed herein. A “radial axis” is normal to the longitudinal axis and extends in a radial direction.

In addition, as used herein, “the longitudinal direction” refers to a direction substantially parallel to the longitudinal axis and “the radial direction” refers to a direction substantially parallel to a radial axis. The term “axial” may be used herein and is synonymous with the term “longitudinal” as used herein.

Also, the terms “proximal” and “distal,” which are used to describe the present atherectomy apparatuses, are used consistently with the description of the exemplary applications (i.e., the particular illustrative examples). Thus, proximal and distal are used in reference to the handle of the atherectomy apparatus.

The terms “upper,” “lower,” “top,” “bottom,” “underside,” “upperside” and the like are used in reference to the illustrated orientation of the embodiment.

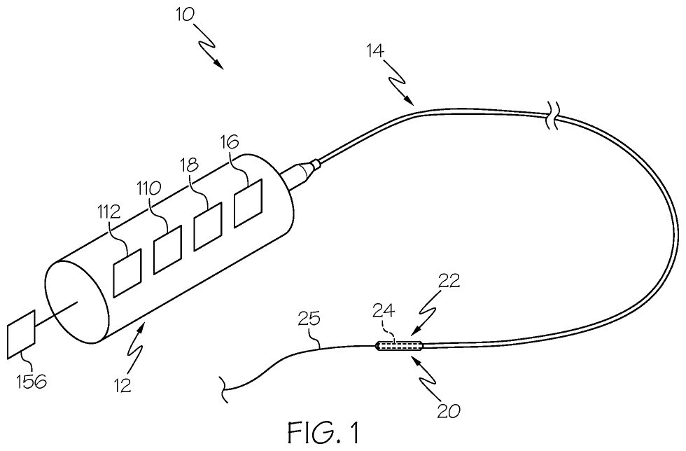

Referring to FIG. 1, an intravascular apparatus 10 includes a handle 12 and an intravascular catheter 14 that extends distally outward from the handle 12. The handle 12 includes an electrical circuit, represented by element 16, that regulates electrical power from a power source 18. The power source 18 may be a battery internal to the handle 12 or be external, such as an outlet, generator, etc. The electrical circuit 16 is electrically coupled to a solenoid-type reciprocating device 20 that is located at a distal end portion 22 of the intravascular catheter 14. The electrical circuit 16 is configured to generate a series of electrical pulses to actuate the solenoid-type reciprocating device 20, as will be described in greater detail below. In some embodiments, the intravascular catheter 14 may also include an anchor balloon 24 at the distal end portion 22. In some embodiments, the intravascular catheter 14 and the solenoid-type reciprocating device 20 may be advanceable over a guidewire 25 and/or through a sheath. As an example, the intravascular apparatus 10 may comprise an over-the-wire intravascular catheter 14.

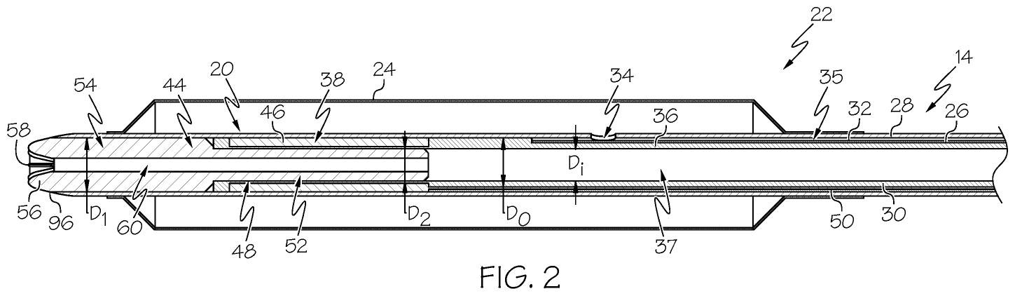

Referring to FIG. 2, a diagrammatic section view of the distal end portion 22 of the intravascular catheter 14 including solenoid-type reciprocating device 20 and anchor balloon 24 is illustrated. The intravascular catheter 14 includes an elongate catheter body 26 that includes an outer catheter body 28 (e.g., a sheath) and an inner catheter body 30. A space 35 between the outer catheter body 28 and the inner catheter body 30 provides an inflation lumen 32 through which a fluid, such as saline, can be delivered to an internal, expandable volume of the anchor balloon 24. For example, the fluid may be delivered from a fluid source, through the inflation lumen 32 and through an aperture 34 that is in communication with the expandable volume for inflating the anchor balloon 24. The anchor balloon 24 may be a compliant balloon, non-compliant balloon or semi-compliant balloon, as determined to be needed for a particular procedure.

The inner catheter body 30 has a sidewall 36 that defines a work lumen 37 that extends axially therethrough from the handle 12 (FIG. 1) beyond a solenoid coil 38. The work lumen 37 may provide a passageway through which the guidewire 25 (FIG. 1) may pass. In some embodiments, the solenoid coil 38 may be located within the sidewall 36 or, in some embodiments, the solenoid coil 38 may be located on the sidewall 36. For example, the solenoid coil 38 may be embedded into the sidewall 36 or attached to an interior surface 40 or an exterior surface 42 of the inner catheter body 30. In another embodiment, the solenoid coil 38 may be located in or attached to a separate tubular structure that is then attached to the inner catheter body 30 to form a distal portion of the inner catheter body 30. Any suitable structure can be used to house or otherwise support the solenoid coil 38 adjacent the work lumen 37. The material that forms the inner catheter body 30 at the solenoid coil 38 may be selected to minimize or even enhance the magnetic field that is formed using the solenoid coil 38.

As can be seen, an outer diameter Dₒ of the inner catheter body 30 may increase in the distal end portion 22. The increase in outer diameter Dₒ can close off the inflation lumen 32 and force the fluid through the aperture 34 to inflate the anchor balloon 24. The increase in outer diameter Dₒ can also provide additional material thickness in which to at least partially embed the solenoid coil 38. In some embodiments, an inner diameter Dᵢ of the work lumen 37 remains substantially constant over the length of the distal end portion 22. A substantially constant inner diameter Dᵢ can reduce interference between the inner catheter body 30 and the guidewire 25 (FIG. 1) passing therethrough.

The solenoid-type reciprocating device 20 is located in the distal end portion 22 of the inner catheter body 30. The solenoid-type reciprocating device 20 includes the solenoid coil 38 and a distal tip member 44. The solenoid coil 38 includes a wire 46 that is wound into a helix and arranged to produce a uniform magnetic field through a coil aperture 48 in order to move the distal tip member 44 linearly back-and-forth in a reciprocating fashion when an electric current is passed through the wire 46. The opposite ends of the solenoid coil 38 may be connected to lead wires 50 that are electrically connected to the electrical circuit 16 (FIG. 1). The lead wires 50 may extend longitudinally in the space 35 between the outer catheter body 28 and inner catheter body 30. In some embodiments, the lead wires 50 may travel through a separately provided lumen and/or be printed onto the outer and/or inner catheter body 28, 30 as a flex circuit.

The wire 46 for the solenoid coil 38 may be an electrically insulated, electrically conductive copper wire that has a layer of an insulated coating. The gauge and length of the wire 46 as well as characteristics of the solenoid coil 38, such as number of turns and turn density, may be selected to provide the desired electric field. The wire may be wound on a spool formed of a suitable non-ferromagnetic material.

The distal tip member 44 has a proximal armature section 52 and a distal working end section 54. The proximal armature section 52 is sized to be slidably received within the coil aperture 48. The proximal armature section 52 includes or is formed of a ferromagnetic material so as to be influenced by the magnetic field produced by the solenoid coil 38. For example, the proximal armature section 52 may be formed of or include a permanent magnet providing polarity. In some embodiments, the proximal armature section 52 may be formed of or include a magnetized ferromagnetic material such as used to form only part of the proximal armature section 52, such as a core. In some embodiments, a ferromagnetic material may be used that is not magnetized and a spring or other actuator may be used to push or pull the distal tip member back to an initial position. Operation of the solenoid-type reciprocating device 20 is described in greater detail below.

The distal working end section 54 may be integrally formed with the proximal armature section 52. In other embodiments, the distal working end section 54 may be formed separately from the proximal armature section 52 and then connected thereto. In the illustrated example, the distal working end section 54 extends both distally and radially outward from the proximal armature section 52 providing the distal working end section 54 having an outer diameter D₁ that is greater than an outer diameter D2 of the proximal armature section 52. In this regard, the distal working end section 54 may have a width that is greater than a width of the coil aperture 48, which can provide a stop that limits linear motion of the distal working end section 54 into the coil aperture 48. The distal working end section 54 extends distally to an impact end 56 having a plurality of spaced ridges 58 configured to deliver a focused impact to the hard areas of plaque to break up the plaque build-up, as will be described in greater detail below. The distal tip member 44 also has a guidewire lumen 60 through which the guidewire 25 (FIG. 1) can pass through and out of the end 56.

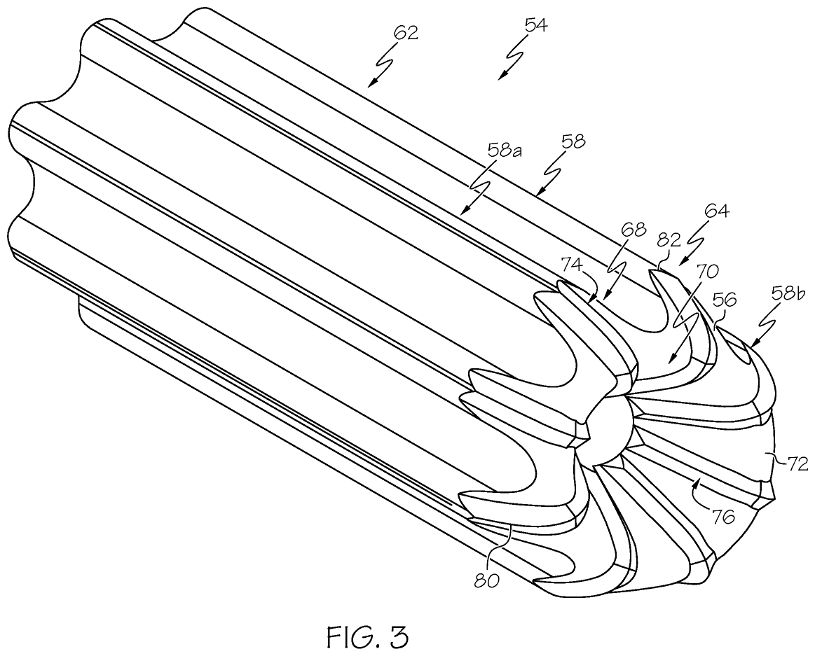

FIG. 3 illustrates a more detailed, perspective view of the distal working end section 54 in isolation. In this embodiment, the distal working end section 54 has a generally cylindrical portion 62 and a distal cap portion 64 that forms the annular impact end 56. The plurality of ridges 58 are circumferentially spaced apart and extend longitudinally over lengths of both the cylindrical portion 62 and the distal cap portion 64.

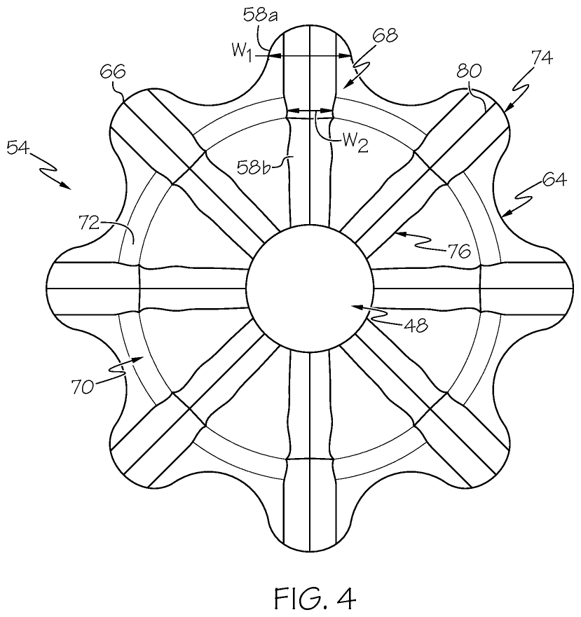

Referring also to FIG. 4, the plurality of ridges 58 includes longitudinal ridges 58a and radial ridges 58b. The longitudinal ridges 58a extend over the cylindrical portion 62 and the radial ridges 58b extend over the distal cap portion 64. As can be most clearly seen in FIG. 4, the longitudinal ridges 58a have a width W₁ that is greater than a width W2 of the radial ridges 58b. Further, the radial ridges 58b may be aligned with the longitudinal ridges 58a such that each radial ridge 58b intersects an apex 66 of each longitudinal ridge 58a.

The distal cap portion 64 includes an exterior extending portion 68 and an interior extending portion 70. The exterior extending portion 68 and interior extending portion 70 meet at an edge 72 that forms part of the impact end 56. The radial ridges 58b extend continuously over both the exterior extending portion 68, the interior extending portion 70 and the edge 72. Thus, the radial ridges 58b each have an exterior extending portion 74, an interior extending portion 76 and a bend portion 78 that connects the exterior extending portion 74 and the interior extending portion 76. The interior extending portion 76 terminates at the coil aperture 48, while the exterior extending portion 74 terminates at an opposite edge 82 of the distal cap portion 64.

The longitudinal ridges 58a and the radial ridges 58b may have any suitable cross-sectional shape. In the illustrated example, the longitudinal ridges 58a have a more rounded, arcuate cross-sectional shape and the radial ridges 58b have a more triangular cross-sectional shape, coming to a point apex 80 that can be used to further concentrate the forces applied by the distal tip member 44.

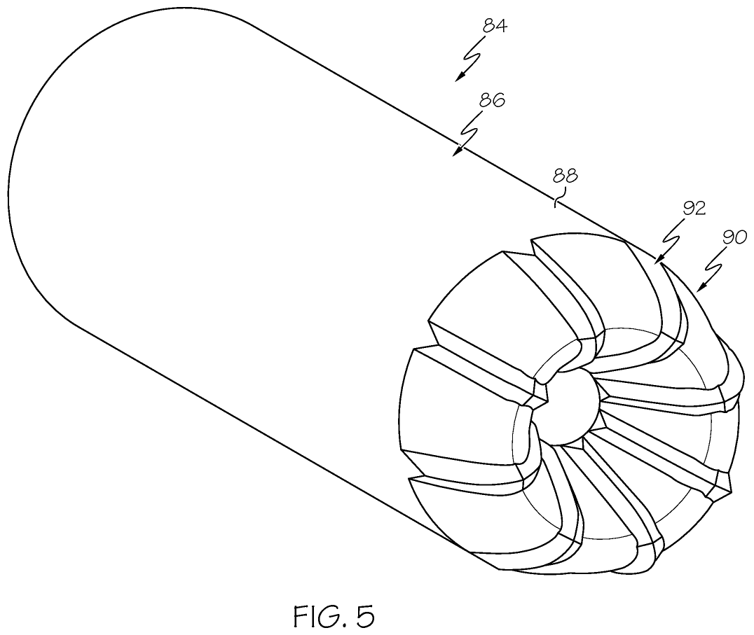

Referring briefly to FIG. 5, another embodiment of a distal working section 84 includes a cylindrical portion 86 without longitudinal ridges. Instead, the cylindrical portion 86 has a smooth exterior surface 88 without any ridges and a distal cap portion 90 with radial ridges 92 similar to those described above.

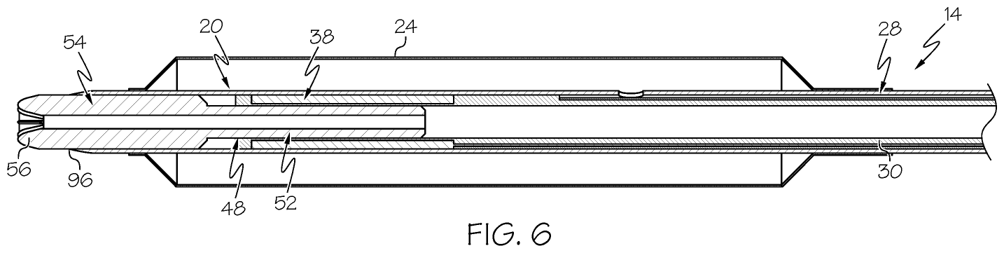

FIG. 2 illustrates the solenoid-type reciprocating device 20 in a retracted configuration with the proximal armature section 52 of the distal tip member 44 fully retracted into the coil aperture 48. In this example, the distal working end section 54 may be sized such that the impact end 56, in the fully retracted configuration, is only slightly spaced outward from a terminal end 96 of the outer catheter body 28 (e.g., no more than about 0.025 inch (0.6 mm), such as no more than about 0.01 inch (0.25 mm)). Referring now to FIG. 6, the solenoid-type reciprocating device 20 is illustrated in an extended configuration with the proximal armature section 52 fully extended out of the coil aperture 48. In this example, the distal working end section 54 may be sized such that the impact end 56, in the fully extended configuration, moves distally relative to the terminal end 96 of the outer catheter body 28 a predetermined distance (e.g., between about 0.025 inch (0.6 mm) and about 0.1 inch (2.54 mm)).

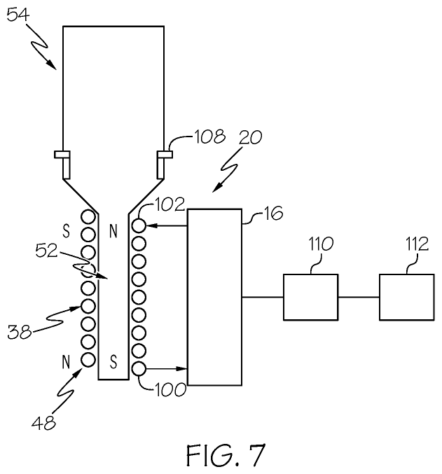

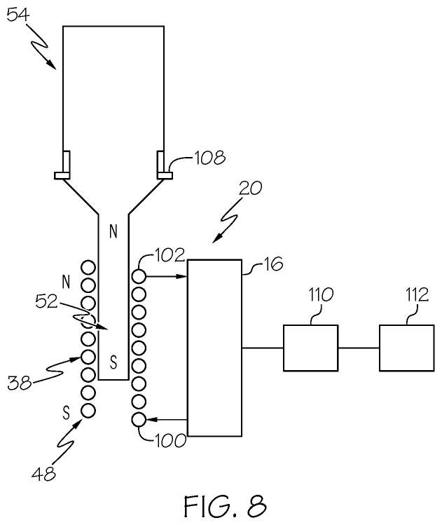

Referring to FIGS. 7 and 8, operation of the solenoid-type reciprocating device 20 is shown and now described. With the solenoid-type reciprocating device 20 in a desired location, the guidewire 25 (FIG. 1) may be retracted to a position proximal of the distal tip member 44. FIG. 7 illustrates the solenoid-type reciprocating device in the retracted configuration with the proximal armature section 52 of the distal tip member 44 fully retracted into the coil aperture 48. In this example, the proximal armature section 52 includes or may be formed of a magnetic material providing N and S poles. The electrical circuit 16 forms a switching device that is configured to switch the polarity at opposite ends 100 and 102 of the solenoid coil 38 in an alternating fashion to provide a first energized state (FIG. 6) and a second energized state (FIG. 7). In the first energized state, as shown by FIG. 6, the end 100 has a first polarity N and the second end 102 has a second polarity S. In the second energized state, as shown by FIG. 7, the end 100 has the second polarity S and the end 102 has the first polarity N. In the first energized state, the N and S poles of the solenoid coil 38 attract the S and N poles of the proximal armature section 52 thereby retracting the proximal armature section 52 and the distal working end section 54 together. In the second energized state, the N and S poles of the solenoid coil 38 repel the N and S poles of the proximal armature section 52 thereby extending the proximal armature section 52 and distal working end section 54 together. While a single solenoid coil is illustrated, multiple solenoid coils may be used. A stop 108 may be provided to limit linear movement of the distal tip member 44 toward one or both of the retracted and extended configurations.

The electrical circuit 16 may comprise any components for providing and regulating the reverse magnetic switching needed to drive the distal tip member 44 in a reciprocating fashion. The electrical circuit 16 may include converters, capacitors, diodes, transistors, inductors, bridges, etc. to achieve switching output polarity. A control unit 110 may be used to control operation of the electrical circuit 16. In some embodiments, a user input 112 may be provided that allows a user to activate, deactivate and otherwise control operation of the solenoid-type reciprocating device 20. For example, the user input 112 may include a control that allows for adjustment of reciprocating frequencies (e.g., between zero and 100 Hz).

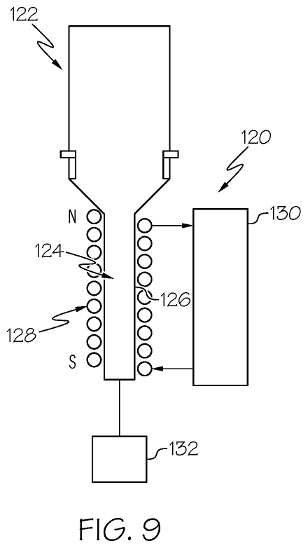

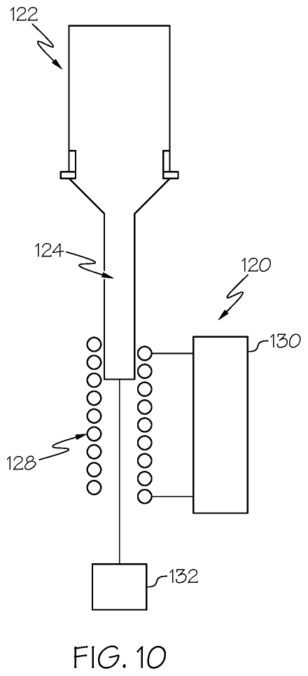

While the above-described embodiments use reverse magnetic switching, solenoid-type reciprocating devices may use a mechanical actuator, such as a spring and an ON/OFF arrangement to provide the reciprocal motion. FIGS. 9 and 10 illustrate an example of such a solenoid-type reciprocating device 120 that includes many of the described components including a distal tip member 122 including a proximal armature section 124 that is received within a coil aperture 126 of a solenoid coil 128. In this embodiment, the proximal armature section 124 may be formed of a ferromagnetic material that is not magnetized or include a permanent magnet. Instead, each time the solenoid coil 128 is provided an electric current using an electrical circuit 130, the distal tip member 122 is attracted toward the solenoid coil 128. To this end, the electrical circuit 130 only needs to provide a voltage of a single polarity to move the distal tip member 122 toward the retracted configuration.

Because the distal tip member 122 is only attracted toward the solenoid coil 128 when current is provided to the solenoid coil 128, an actuator 132 is used to push the distal tip member 122 toward the extended configuration, as shown by FIG. 10. The actuator 132 may be, for example, a spring or other suitable device.

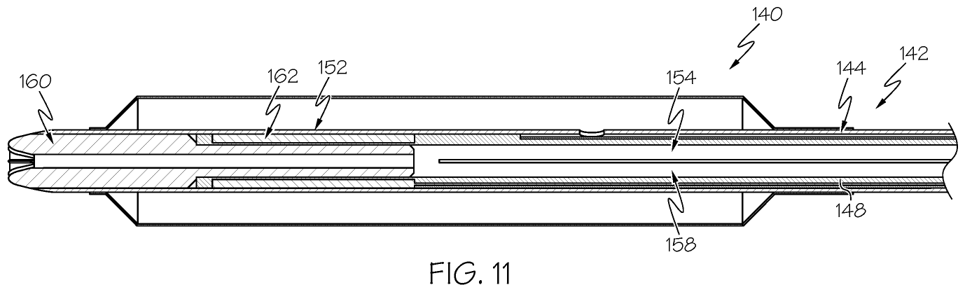

Referring now to FIG. 11, another embodiment of an intravascular apparatus 140 includes many of the components described above including an intravascular catheter 142 that includes an elongate catheter body 144 including an outer catheter body 146 and an inner catheter body 148. As above, a distal end portion 150 of the catheter body 144 is provided with a solenoid-type reciprocating device 152. In this embodiment, a vacuum aspiration pathway 154 is provided through an upper portion of inner catheter body 148. The vacuum pathway 154 may be in communication with a vacuum source 156 (FIG. 1) to aspirate the blood vessel lumen during or after plaque is broken-up using the solenoid-type reciprocating device 152. A work lumen 158 is located below the vacuum pathway 154 and separated therefrom. A separate work lumen 158 can allow a guidewire to remain in place through most of the length of the intravascular catheter 142 and to retract to just proximal of a distal tip member 160 when vacuum aspiration is provided and the distal tip member is being actuated by solenoid coil 162.

The above-described intravascular apparatuses utilize a solenoid-type reciprocating device in order to rapidly reciprocate a distal tip member for breaking up and removing plaque from calcified blood vessels. The distal tip member is driven linearly into the plaque via an electromagnetic force, actuating with short, rapid strokes. The distal tip member may be provided with ridges and/or points that focus the force of the distal tip member into the plaque, which can crack and penetrate surfaces of the plaque in multiple locations simultaneously. The intravascular apparatuses can atherectomize while crossing the lesion providing blood flow through the lumen. An anchoring balloon can be used to keep the intravascular catheter in place during distal tip member actuations. The anchoring balloon can also be used to expand the lumen of the blood vessel. The above-described intravascular apparatuses can reduce the steps needed in an intravascular crossing and atherectomy procedure. For example, a vacuum lumen can be provided for aspiration and the guide wire can be retracted only enough to remove it from the distal tip member during an aspiration procedure.

Embodiments can be described with reference to the following numbered clauses, with certain features laid out in the dependent clauses:
Clause 1: An intravascular apparatus, comprising: an electrical circuit configured to generate a series of electrical pulses; and an intravascular catheter that includes: an elongate catheter body having a distal end portion and a distal end at a distal terminus of the distal end portion; and a solenoid-type reciprocating device located at the distal end portion of the elongate catheter body, the solenoid-type reciprocating device having a coil and a distal tip member, the coil being electrically coupled to the electrical circuit, the distal tip member having a proximal armature section and a distal working end section, the coil defining a coil aperture configured to slidably receive the proximal armature section, wherein the proximal armature section is configured to axially reciprocate relative to the coil when the coil is energized by the series of electrical pulses generated by the electrical circuit, and the distal working end section is configured to axially reciprocate from the distal end of the elongate catheter body, in unison with the axial reciprocation of the proximal armature section.
Clause 2: The intravascular apparatus according to clause 1, wherein the elongate catheter body has a side wall and a work lumen, wherein the coil of the solenoid-type reciprocating device is located on or in the side wall of the elongate catheter body, and the work lumen is configured to slidably carry the distal tip member of the solenoid-type reciprocating device.
Clause 3: The intravascular apparatus according to clause 1 or 2, comprising an anchor balloon affixed to the distal end portion of the elongate catheter body.
Clause 4: The intravascular apparatus according to clause 3, wherein the anchor balloon is configured to surround at least a portion of the distal end portion of the elongate catheter body.
Clause 5: The intravascular apparatus according to clause 3 or 4, wherein the elongate catheter body has an inflation lumen that is in fluid communication with the anchor balloon.
Clause 6: The intravascular apparatus according to any of clauses 1-5, wherein the distal tip member has a guidewire aperture.
Clause 7: The intravascular apparatus of clause 6, wherein the elongate catheter body comprises a vacuum pathway that extends therethrough and provides a reduced pressure through the guidewire lumen to aspirate a blood vessel lumen.
Clause 8: The intravascular apparatus according to any of clauses 1-7, wherein the distal working end section of the distal tip member has a plurality of circumferentially spaced ridges configured to deliver focused impact when reciprocated.
Clause 9: The intravascular apparatus according to clause 8, wherein the distal working end section has a cylindrical portion and a distal cap portion, therein the cylindrical portion has a smooth exterior surface and the distal cap portion includes the plurality of circumferentially spaced ridges.
Clause 10: The intravascular apparatus according to clause 8, wherein the distal working end section has a cylindrical portion and a distal cap portion, therein both the cylindrical portion and the distal cap portion includes the plurality of circumferentially spaced ridges.
Clause 11: The intravascular apparatus according to any of clauses 8-10, wherein the distal working end section has an annular impact end, and wherein each of the plurality of circumferentially spaced ridges distally extends to the annular impact end to form a plurality of impact protrusions at the annular impact end.
Clause 12: An intravascular catheter, comprising: an elongate catheter body having a distal end portion and a distal end at a distal terminus of the distal end portion; and a solenoid-type reciprocating device located at the distal end portion of the elongate catheter body, the solenoid-type reciprocating device having a coil and a distal tip member, the distal tip member having a proximal armature section and a distal working end section, the coil defining a coil aperture configured to slidably receive the proximal armature section, wherein the proximal armature section is configured to axially reciprocate relative to the coil and the distal working end section is configured to axially reciprocate from the distal end of the elongate catheter body.
Clause 13: The intravascular catheter according to clause 12, wherein the elongate catheter body has a side wall and a work lumen, wherein the coil of the solenoid-type reciprocating device is located on or in the side wall of the elongate catheter body, and the work lumen is configured to slidably carry the distal tip member of the solenoid-type reciprocating device.
Clause 14: The intravascular catheter according to clause 12 or 13, comprising an anchor balloon affixed to the distal end portion of the elongate catheter body.
Clause 15: The intravascular catheter according to clause 14, wherein the anchor balloon is configured to surround at least a portion of the distal end portion of the elongate catheter body.
Clause 16: The intravascular catheter according to clause 14 or 15, wherein the elongate catheter body has an inflation lumen that is in fluid communication with the anchor balloon.
Clause 17: The intravascular catheter according to any of clauses 12-16, wherein the distal tip member has a guidewire aperture.
Clause 18: The intravascular catheter of clause 17, wherein the elongate catheter body comprises a vacuum pathway that extends therethrough and provides a reduced pressure through the guidewire lumen to aspirate a blood vessel lumen.
Clause 19: The intravascular catheter according to any of clauses 12-18, wherein the distal working end section of the distal tip member has a plurality of circumferentially spaced ridges configured to deliver focused impact when reciprocated.
Clause 20: The intravascular catheter according to any clause 19, wherein the distal working end section has a cylindrical portion and a distal cap portion, therein the cylindrical portion has a smooth exterior surface and the distal cap portion includes the plurality of circumferentially spaced ridges.
Clause 21: The intravascular catheter according to clause 19, wherein the distal working end section has a cylindrical portion and a distal cap portion, therein both the cylindrical portion and the distal cap portion includes the plurality of circumferentially spaced ridges.
Clause 22: The intravascular catheter according to any of clauses 19-21, wherein the distal working end section has an annular impact end, and wherein each of the plurality of circumferentially spaced ridges distally extends to the annular impact end to form a plurality of impact protrusions at the annular impact end.

It will be apparent to those skilled in the art that various modifications and variations can be made to the embodiments described herein without departing from the spirit and scope of the claimed subject matter. Thus it is intended that the specification cover the modifications and variations of the various embodiments described herein provided such modification and variations come within the scope of the appended claims and their equivalents.

1. An intravascular apparatus, comprising:
an electrical circuit configured to generate a series of electrical pulses; and
an intravascular catheter that includes:
an elongate catheter body having a distal end portion and a distal end at a distal terminus of the distal end portion; and
a solenoid-type reciprocating device located at the distal end portion of the elongate catheter body, the solenoid-type reciprocating device having a coil and a distal tip member, the coil being electrically coupled to the electrical circuit, the distal tip member having a proximal armature section and a distal working end section, the coil defining a coil aperture configured to slidably receive the proximal armature section,
wherein the proximal armature section is configured to axially reciprocate relative to the coil when the coil is energized by the series of electrical pulses generated by the electrical circuit, and the distal working end section is configured to axially reciprocate from the distal end of the elongate catheter body, in unison with the axial reciprocation of the proximal armature section.

2. The intravascular apparatus according to claim 1, wherein the elongate catheter body has a side wall and a work lumen, wherein the coil of the solenoid-type reciprocating device is located on or in the side wall of the elongate catheter body, and the work lumen is configured to slidably carry the distal tip member of the solenoid-type reciprocating device.

3. The intravascular apparatus according to claim 1, comprising an anchor balloon affixed to the distal end portion of the elongate catheter body.

4. The intravascular apparatus according to claim 3, wherein the anchor balloon is configured to surround at least a portion of the distal end portion of the elongate catheter body.

5. The intravascular apparatus according to claim 3, wherein the elongate catheter body has an inflation lumen that is in fluid communication with the anchor balloon.

6. The intravascular apparatus according to claim 1, wherein the distal tip member has a guidewire aperture.

7. The intravascular apparatus of claim 6, wherein the elongate catheter body comprises a vacuum pathway that extends therethrough and provides a reduced pressure through the guidewire lumen to aspirate a blood vessel lumen.

8. The intravascular apparatus according to claim 1, wherein the distal working end section of the distal tip member has a plurality of circumferentially spaced ridges configured to deliver focused impact when reciprocated.

9. The intravascular apparatus according to claim 8, wherein the distal working end section has a cylindrical portion and a distal cap portion, therein the cylindrical portion has a smooth exterior surface and the distal cap portion includes the plurality of circumferentially spaced ridges.

10. The intravascular apparatus according to claim 8, wherein the distal working end section has a cylindrical portion and a distal cap portion, therein both the cylindrical portion and the distal cap portion includes the plurality of circumferentially spaced ridges.

11. The intravascular apparatus according to claim 8, wherein the distal working end section has an annular impact end, and wherein each of the plurality of circumferentially spaced ridges distally extends to the annular impact end to form a plurality of impact protrusions at the annular impact end.

12. An intravascular catheter, comprising:
an elongate catheter body having a distal end portion and a distal end at a distal terminus of the distal end portion; and
a solenoid-type reciprocating device located at the distal end portion of the elongate catheter body, the solenoid-type reciprocating device having a coil and a distal tip member, the distal tip member having a proximal armature section and a distal working end section, the coil defining a coil aperture configured to slidably receive the proximal armature section, wherein the proximal armature section is configured to axially reciprocate relative to the coil and the distal working end section is configured to axially reciprocate from the distal end of the elongate catheter body.

13. The intravascular catheter according to claim 12, wherein the elongate catheter body has a side wall and a work lumen, wherein the coil of the solenoid-type reciprocating device is located on or in the side wall of the elongate catheter body, and the work lumen is configured to slidably carry the distal tip member of the solenoid-type reciprocating device.

14. The intravascular catheter according to claim 12, comprising an anchor balloon affixed to the distal end portion of the elongate catheter body.

15. The intravascular catheter according to claim 14, wherein the anchor balloon is configured to surround at least a portion of the distal end portion of the elongate catheter body.

16. The intravascular catheter according to claim 14, wherein the elongate catheter body has an inflation lumen that is in fluid communication with the anchor balloon.

17. The intravascular catheter according to claim 12, wherein the distal tip member has a guidewire aperture.

18. The intravascular catheter of claim 17, wherein the elongate catheter body comprises a vacuum pathway that extends therethrough and provides a reduced pressure through the guidewire lumen to aspirate a blood vessel lumen.

19. The intravascular catheter according to claim 12, wherein the distal working end section of the distal tip member has a plurality of circumferentially spaced ridges configured to deliver focused impact when reciprocated.

20. The intravascular catheter according to claim 19, wherein the distal working end section has a cylindrical portion and a distal cap portion, therein the cylindrical portion has a smooth exterior surface and the distal cap portion includes the plurality of circumferentially spaced ridges.

21. The intravascular catheter according to claim 19, wherein the distal working end section has a cylindrical portion and a distal cap portion, wherein both the cylindrical portion and the distal cap portion includes the plurality of circumferentially spaced ridges.

22. The intravascular catheter according to claim 19, wherein the distal working end section has an annular impact end, and wherein each of the plurality of circumferentially spaced ridges distally extends to the annular impact end to form a plurality of impact protrusions at the annular impact end.
